(19) United States
(12) Patent Application Publication
(10) Pub. No.: US 2023/0230274 A1
(43) Pub. Date: Jul. 20, 2023

(54) BACK IMAGE INTERPRETATION ASSISTANCE DEVICE, INTERPRETATION ASSISTANCE METHOD, AND PROGRAM

(71) Applicant: JAPANESE ORGANIZATION FOR MEDICAL DEVICE DEVELOPMENT, INC., Tokyo (JP)

(72) Inventors: Yuta Crawford SHISHIME, Tokyo (JP); Masakatsu NOGUCHI, Tokyo (JP); Masahiro SHINODA, Tokyo (JP)

(73) Assignee: JAPANESE ORGANIZATION FOR MEDICAL DEVICE DEVELOPMENT, INC., Tokyo (JP)

(21) Appl. No.: 17/928,601

(22) PCT Filed: Apr. 30, 2021
(86) PCT No.: PCT/JP2021/017242

§ 371 Date: Nov. 29, 2022

(30) Foreign Application Priority Data
May 29, 2020 (JP) 2020-094333

(51) Int. Cl. G06T 7/68; G06T 7/543; G06T 11/00; G06T 7/55; G06T 7/00; A61B 5/00

(52) U.S. Cl. CPC G06T 7/68; G06T 7/543; G06T 11/00; G06T 7/55; G06T 7/0012; A61B 5/4561; G06T 2207/30012; G06T 2207/30172

(57) ABSTRACT

[Problem] To perform interpretation assistance of a back portion image in real time using a general-purpose computer for any case of scoliosis.

[Solution] An interpretation assistance apparatus includes a center line creating unit that creates a center line C in a back portion image of a subject, a measurement interval designating unit that accepts designation of an arbitrary measurement interval I along the center line C, a measurement width designating unit that accepts designation of an arbitrary measurement width W toward intersecting directions of the center line C, and a measurement point coordinate obtaining unit that obtains depth coordinates of respective measurement points that are apart from the center line on both the right and left sides by the measurement width W at every measurement interval I.

### TECHNICAL FIELD

The present invention relates to an apparatus, a method, and a program for assisting interpretation of a back portion image of a subject. The present invention is used as a medical device mainly for performing an examination of scoliosis.

### BACKGROUND ART

Scoliosis frequently occurs from the upper grades of elementary school to junior high school years, and it is also reported that the prevalence rate in girls of the ages from 13 to 14 is 2.5%. Deformity of the spine due to scoliosis causes low back pain, back portion pain, respiratory dysfunction, and the like in some cases. Since surgery at a serious level puts a large burden on a patient, and a hospitalization cost is expensive, it is considered important to detect scoliosis early and suppress its progression. In particular, by the Bracing in Adolescent Idiopathic Scoliosis Trial (BrAIST) study performed in 2013 with support of the U.S. National Institutes of Health, high effectivity of a bracing treatment for patients with mild or moderate scoliosis has been proven, and early detection of scoliosis has been reaffirmed. In Japan, according to the School Health and Safety Act, it is compulsory to conduct a scoliosis examination when starting school and at a periodic school medical examination.

Here, the current scoliosis examination is constituted of a primary examination (school medical examination) and a secondary examination (detailed inspection). The primary examination targets all students and is performed with a visual examination, palpation, or a moire test mainly by school doctors, medical examination providers, and practitioners. In a case where it is determined there that there is a suspicion of scoliosis, a subject proceeds to the secondary examination, and X-ray imaging is conducted to make a confirmed diagnosis of scoliosis. However, since approximately 90% of the primary examinations are performed with a visual examination and palpation instead of a moire test, oversight of scoliosis (low sensitivity) and high false positivity (low specificity) have been problems. Regardless of such disadvantages, in the primary examination, a moire test having objectivity and high recordability is not conducted. As one reason for that, difficulty of interpreting a moire image output by a special moire test device has been pointed out by scoliosis specialists and the like.

In the moire test, a medical examination provider or the like captures a back portion of a test subject using the special moire test device. Accordingly, a surface shape of the back portion of the test subject is captured as a contour image (that is, moire image). Subsequently, the medical examination provider or the like interprets features, such as this moire image being right-left asymmetric on the back portion of the subject, and thereby determines whether the subject is to take the secondary examination (detailed inspection). However, currently, in order to measure how many fringes of fringe patterns in the moire image of the back portion of the subject are different between the right and left, a skilled image interpreter uses a ruler or the like to imagine a center line connecting the cervical spine on the head side of the subject and the sacral spine on the buttock side, and in the meantime, slides the ruler to measure a right-left asymmetric degree. Accordingly, while it becomes possible to interpret the moire image objectively to some extent, the interpreting time per piece is nearly one minute, proficiency is required to acquire an interpretation skill, and a record of where the center line has been actually assumed to determine a right-left difference does not remain.

Here, in Patent Document 1, an evaluation system of scoliosis that allows highly accurately performing a quantitative evaluation of scoliosis with ease and at low cost is proposed. The evaluation system described in Patent Document 1 detects a center line in a perpendicular direction of a back portion of a subject based on three-dimensional data of the back portion of the subject, and calculates a height difference between uneven peak positions on the right and left of the center line in a designated feature portion. Accordingly, a distortion degree of the feature portion is considered to be obtainable with ease and at low cost. In addition, in Patent Document 2, a scoliosis diagnosis assistance device, a scoliosis diagnosis assistance method, and a program that can assist early detection of scoliosis are proposed. The scoliosis diagnosis assistance device described in Patent Document 2 obtains distribution of deviations between a three-dimensional shape of a back of a subject and a three-dimensional shape in a mirror-symmetry relationship with respect to a sagittal plane of the three-dimensional shape, and outputs this as diagnosis assistance information on scoliosis.

Patent Document 1: Pamphlet of WO2013/081030

### SUMMARY OF THE INVENTION

### Problems to be Solved by the Invention

The evaluation system in Patent Document 1 premises that a position where the spine exists in a back portion of a human body is a bottom in a recessed state and projected peak positions always exist on both the right and left sides of the center line passing above this spine. However, in actual scoliosis, the spine does not necessarily have a bottom in a recessed state thereon, and on the contrary, there is a case where the spine has a projected state thereon, or a case where a projected peak position exists only on either the right or left of the spine. In such cases, there has been a problem that a height difference between both the right and left sides of the center line cannot be appropriately calculated by the evaluation system in Patent Document 1.

In addition, the scoliosis diagnosis assistance device in Patent Document 2 obtains three principal axes by principal component analysis of a back mesh (three-dimensional shape indicated by back shape information) of a back of a subject, obtains a sagittal plane based on two of the axes, identifies corresponding points in the back mesh (three-dimensional shape indicated by back shape information) and a mirror-symmetric mesh (three-dimensional shape indicated by mirror-symmetry information) that have a mirror-symmetry relationship with respect to this sagittal plane, and obtains respective distances between the corresponding points as distribution of deviations. However, in actual scoliosis, since the spine of the subject is curved, three-dimensional distribution on a back portion is not uniform. Therefore, in a method in which a device obtains a reference sagittal plane and distances between corresponding points analytically case by case, as with the scoliosis diagnosis assistance device in Patent Document 2, there has been a problem that, in some cases, these cannot be appropriately calculated within a determined time using a general-purpose computer in, for example, a school medical examination where several thousand pieces of back portion images are required to be processed at once.

Therefore, the present invention has a main object to provide an interpretation assistance technique of a back portion image that is usable in real time using a general-purpose computer for any case of scoliosis.

### Solutions to the Problems

The inventors of the present invention seriously examined means to achieve the above-described object and obtained knowledge that, by enabling creating a predetermined center line in a back portion image of a subject, and arbitrarily designating a measurement interval and a measurement width for setting measurement points where a height difference between both the right and left sides of this center line was measured, even when projected peak positions did not exist on both the right and left sides of the center line, the height difference between the measurement points on both the right and left sides of the center line could be calculated. Further, the present inventors conceived that, based on the above-described knowledge, an apparatus and a method that were generally usable for any case of scoliosis could be provided, and completed the present invention. To be specific, the present invention includes the following configurations and steps.

A first aspect of the present invention relates to an interpretation assistance apparatus of a back portion image of a subject. The interpretation assistance apparatus according to the present invention includes a center line creating unit, a measurement interval designating unit, a measurement width designating unit, and a measurement point coordinate obtaining unit. The center line creating unit creates a center line extending in an up-down direction in the back portion image of the subject. As described later, the center line may be one that is automatically created so as to pass through each of an arbitrary plurality of plots whose designation has been accepted. In addition, similarly to Patent Document 1, for example, the center line may be one that is automatically created based on three-dimensional data of a back portion of the subject. Specifically, since a position where the spine exists in a back portion of a human body has a recessed state, predetermined positions that are approximate to the center and are a bottom in a recessed state in the three-dimensional data of the back portion in an uneven state may be connected to form the center line. The measurement interval designating unit accepts designation of an arbitrary measurement interval along the center line from an operator of the apparatus. All the measurement intervals may be equally spaced intervals, or the measurement interval may be able to be minutely designated individually or for each predetermined group. As a designation method of the measurement interval, for example, an interval length (such as millimeter) may be designated, or the number of intervals to be set on the center line may be designated. The measurement width designating unit accepts designation of an arbitrary measurement width toward intersecting directions of the center line from the operator of the apparatus. The measurement widths may have equally spaced intervals on the right and left of the center line, or may be able to be designated separately on the right and left. In addition, the same width may be set at all the measurement intervals, or the width may be able to be minutely designated individually for each measurement interval or for each predetermined group. In addition, the “intersecting directions” of the center line include not only a direction perpendicular to the center line, but also directions extending slightly inclining without being perpendicular to the center line. The measurement coordinate obtaining unit obtains (at least) depth coordinates of respective measurement points that are apart from the center line on both the right and left sides by the measurement width at every measurement interval. The depth coordinate (depth direction) refers to, in a case where the back portion image of the subject is assumed to be an x-y plane in an xyz three-dimensional orthogonal coordinate system, a z-axis direction perpendicular to this plane. Note that, in the present application, an x-axis direction also refers to a right-left direction, a y-axis direction to the up-down direction, and the z-axis direction to the depth direction. Thus, in the present invention, the measurement points of a height difference do not depend on projected peak positions, and the operator can arbitrarily designate the measurement interval and the measurement width to set these measurement points. Therefore, in the present invention, the height difference between the measurement points on both the right and left sides of the center line can always be calculated. At that time, with the present invention, since a reference surface or distances between corresponding points on the right and left do not need to be obtained analytically case by case, a processing load of a computer can be reduced. Accordingly, even in a case where a large amount of scoliosis cases exist, measurement results can be obtained in real time using a general-purpose computer.

The interpretation assistance apparatus according to the present invention may further include a plot designating unit. The plot designating unit accepts designation of an arbitrary plurality of plots in the back portion image of the subject. The number of plots needs to be at least two, and can also be three or more, or four or more. In this case, the center line creating unit only needs to create the center line so as to pass through the designated plurality of plots in the back portion image. Thus, the operator can designate arbitrary plots, and the interpretation assistance apparatus automatically creates the center line that passes through these plots. Accordingly, the center line as a reference of the measurement points can be flexibly and easily created.

The interpretation assistance apparatus according to the present invention may further include a right-left difference calculating unit. The right-left difference calculating unit calculates a right-left difference between the depth coordinates of the paired right and left measurement points arranged across the center line at every measurement interval. The depth coordinate is a z-coordinate in a case where the back portion image is assumed to be the x-y plane, that is, a coordinate value indicating an uneven height of the back portion of the subject. It means that the larger the right-left difference between the depth coordinates of the paired right and left measurement points is, the wider a curve is at those measurement points. Thus, by automatically calculating the right-left difference between the depth coordinates of the right and left measurement points, the operator can quantitatively obtain a curve degree.

The interpretation assistance apparatus according to the present invention may further include a curved portion identifying unit. The curved portion identifying unit compares the right-left difference of the paired right and left measurement points at each measurement interval, and thereby identifies a position where the right-left difference is at its maximum as a curved portion where a curve degree of the back portion of the subject is high. Thus, by automatically identifying the curved portion, it is possible to shorten time needed for the operator to interpret the back portion image.

The interpretation assistance apparatus according to the present invention may further include a height difference calculating unit. The height difference calculating unit calculates height differences between depth coordinates of paired right and left measurement points arranged across the center line at every measurement interval and a depth coordinate on the center line between the right and left measurement points. That is, the height difference calculating unit obtains the depth coordinate (A-coordinate) of the left side measurement point, the depth coordinate (B-coordinate) of the right side measurement point, and the depth coordinate (C-coordinate) on the center line between these two measurement points. Then, the height difference between the A-coordinate and the C-coordinate, and the height difference between the B-coordinate and the C-coordinate are each calculated. Thus, by calculating the height differences of the right and left measurement points with respect to the center line, the operator can quantitatively obtain a relative curve degree of the right and left measurement points with respect to the center line.

The interpretation assistance apparatus according to the present invention may further include a three-dimensional data obtaining unit, a moire image converting unit, an integrated image creating unit, and a display unit. The three-dimensional data obtaining unit obtains three-dimensional data of the back portion of the subject. As the three-dimensional data, one that is obtained by a publicly known three-dimensional sensor may be input to the three-dimensional data obtaining unit, or one that is already stored locally or in a storage of a cloud may be read out by the three-dimensional data obtaining unit. The moire image converting unit converts the three-dimensional data into a two-dimensional moire image. The moire image is an image illustrating a surface shape of the back portion of an examiner by contour lines. The integrated image creating unit creates an integrated image associating the three-dimensional data with the moire image. That is, in the integrated image, metadata, such as three-dimensional coordinate values included in the three-dimensional data, is embedded in the moire image, and by designating an arbitrary point in a moire visual image, a three-dimensional coordinate value of the point can be read out. The display unit displays this integrated image as the back portion image on a display screen (display). Thus, by creating the integrated image associating the three-dimensional data with the moire image and displaying it on the display screen, a person familiar with a conventional device that displays a moire image can easily acquire an operation method of the interpretation assistance apparatus according to the present invention. Especially, operations of designating plots in the moire image (in the back portion image) to create the center line, designating the measurement interval along the center line, and designating the measurement width of each measurement interval become easy for a person familiar with the operation of the conventional device.

In a case where a plurality of back portion images of the subject exist, the interpretation assistance apparatus according to the present invention may collectively obtain the depth coordinates of the measurement points in the respective back portion images. That is, the plot designating unit preliminarily accepts designation of plots common to the plurality of images. The measurement interval designating unit preliminarily accepts designation of a measurement interval common to the plurality of images. The measurement width designating unit preliminarily accepts designation of a measurement width common to the plurality of images. The center line creating unit creates a center line that passes through the plots in each of the plurality of images. The measurement point coordinate obtaining unit collectively obtains the depth coordinates in each of the plurality of images. Thus, even in a case where a plurality of back portion images of the subject exist, by batch processing the plurality of images, the interpreting time can be substantially shortened.

A second aspect of the present invention relates to a computer program. The program according to the present invention causes a general-purpose computer to function as the interpretation assistance apparatus according to the first aspect described above.

A third aspect of the present invention relates to an interpretation assistance method of a back portion image. The interpretation assistance method according to the present invention includes a step of creating a center line that passes through the plots in a back portion image of a subject, a step of accepting designation of an arbitrary measurement interval along the center line, a step of accepting designation of an arbitrary measurement width toward intersecting directions of the center line, and a step of obtaining depth coordinates of respective measurement points that are apart from the center line on both the right and left sides by the measurement width at every measurement interval.

### Advantageous Effects of the Invention

With the present invention, an interpretation assistance technique of a back portion image that is generally usable for any case of scoliosis can be provided.

### BRIEF DESCRIPTION OF THE DRAWINGS

FIG. 1 is a block diagram illustrating a function configuration of an interpretation assistance apparatus.

FIG. 2 schematically illustrates an exemplary display screen of the interpretation assistance apparatus.

FIG. 3 schematically illustrates an operation method of the interpretation assistance apparatus.

FIG. 4 schematically illustrates an operation method of the interpretation assistance apparatus.

FIG. 5 illustrates respective areas of the spine in a human body and their names.

FIG. 6(a) indicates a measuring method of a height difference (h) in the prior art (mainly Patent Document 1), and FIG. 6(b) indicates a measuring method of a height difference (h) in the present invention.

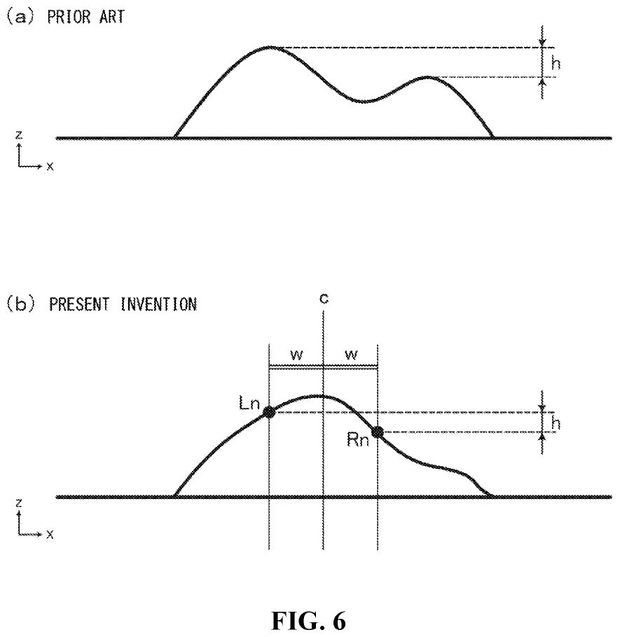

### DETAILED DESCRIPTION OF THE INVENTION

The following describes embodiments for performing the present invention using the drawings. The present invention is not limited to the embodiments described below, and includes modifications appropriately made by those skilled in the art from the following embodiments within an obvious scope.

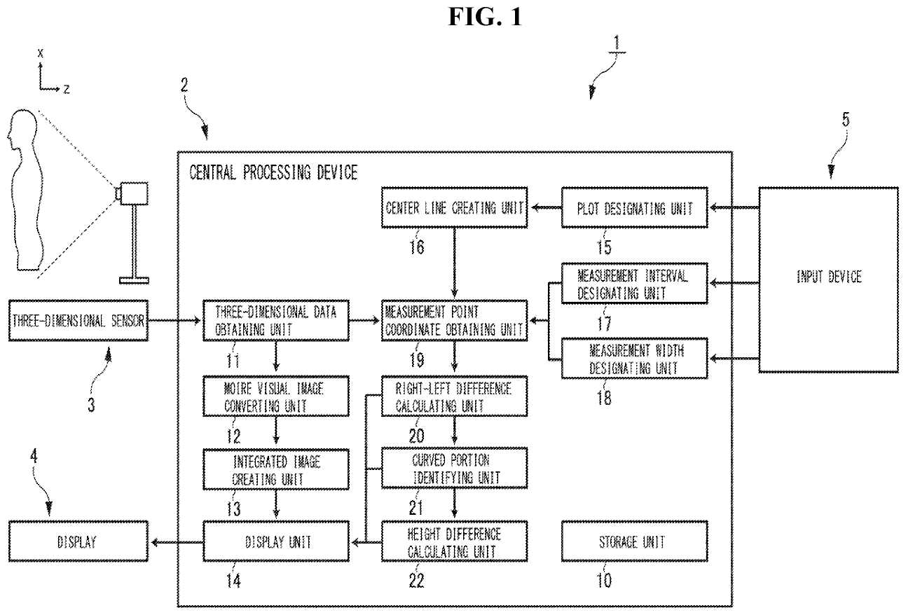

FIG. 1 illustrates a function configuration of one embodiment of an interpretation assistance apparatus 1 according to the present invention. The interpretation assistance apparatus 1 is an apparatus for assisting interpretation of a back portion image of a subject (image capturing the back of the subject in front view). As illustrated in FIG. 1, the interpretation assistance apparatus 1 includes a central processing device 2, and a three-dimensional sensor 3, a display 4, and an input device 5 connected to this central processing device 2. The central processing device 2 causes the display 4 to display the back portion image read by the three-dimensional sensor 3, and accepts input of a predetermined operation and a predetermined setting for reading the back portion image from an operator via the input device 5. Then, the central processing device 2 performs predetermined computation processing based on this input information, and reflects the computation result in the back portion image displayed on the display 4. Thus, the interpretation assistance apparatus 1 basically processes the back portion image so as to allow the operator to easily interpret the back portion image, and displays predetermined assistance information in this back portion image.

The central processing device 2, which is in charge of controlling the entire interpretation assistance apparatus 1, performs predetermined computation processing for interpretation assistance based on information obtained by the three-dimensional sensor 3 and the input device 5, and displays the result thereof on the display 4. As the central processing device 2, a general-purpose computer can be used. The central processing device 2 includes a processor, such as a CPU, and a storage unit 10. By executing a program for interpretation assistance stored in the storage unit 10 by the processor, the general-purpose computer is caused to function as the central processing device 2 specific to the present invention. The central processing device 2 mainly includes the storage unit 10 and functional elements 11 to 22 executed by the processor. The storage function of the storage unit 10 can be achieved by a non-volatile memory, such as an HDD and SDD. In addition, the storage unit 10 may have a function as a memory for writing in or reading out interim progress and the like of computation processing by the processor. The memory function of the storage unit 10 can be achieved by a volatile memory, such as a RAM and a DRAM. Besides that, details of the respective functional elements 11 to 22 included in the central processing device 2 will be described later.

The three-dimensional sensor 3 is a device for capturing a back portion of the subject and obtaining its three-dimensional data. As illustrated in FIG. 1, the three-dimensional sensor 3 is disposed face to face with the back portion of the subject, and by capturing this back portion, obtains an uneven state of the back portion as three-dimensional data. As the three-dimensional sensor 3, a general medical device “3D BACK SCANNER™” manufactured and sold by the present applicant can be employed. The “3D BACK SCANNER™” uses an LED light source, captures the back portion of the subject in three dimensions, and can convert it into a moire pattern visual image to visually depict the back portion symmetry. Besides that, as the three-dimensional sensor 3, for example, one having a publicly known Time of Flight (TOF) system, or one having a laser pattern projection system can be employed. The three-dimensional TOF sensor includes a light source which irradiates an object body (subject) with an invisible inspection light, such as infrared rays, and an image sensor that receives the inspection light reflected on the object. The three-dimensional sensor irradiates the range of the angle of view with the pulse modulated inspection light, measures a phase lag of this pulse using the image sensor, and thereby obtains a round-trip distance to the object. The three-dimensional sensor having the laser pattern projection system irradiates the object body with an infrared ray pattern and obtains a distance image by triangulation. As the three-dimensional sensor having the laser pattern projection system, for example, the Kinect sensor (registered trademark) manufactured by Microsoft Corporation can be employed. Information detected by the three-dimensional sensor 3 is input to the central processing device 2 via a bus, such as an USB.

The display 4 is a device for displaying mainly the back portion image of the subject and the computation result of the central processing device 2. As the display 4, a publicly known display device, such as a liquid crystal display and an organic EL display, need to be employed.

The input device 5 is a device for accepting input of information from the operator to the central processing device 2. The information input via the input device 5 is input to the central processing device 2 via a bus, such as an USB. As the input device 5, various devices used as computer peripheral devices can be employed. Examples of the input device 5 include a touch panel, a keyboard, a computer mouse, a stylus pen, a button, a cursor, and a microphone, but the device is not limited to these. In addition, a touch panel display combining the input device 5 as the touch panel and the display 4 may be employed.

Subsequently, a process of displaying an integrated image that associates the three-dimensional data with the moire image on the display 4 will be described. A three-dimensional data obtaining unit 11 of the central processing device 2 obtains the three-dimensional data of the back portion of the subject. The three-dimensional data is information that records the uneven state of the back portion of the subject as, for example, xyz three-dimensional coordinate values. Therefore, with the three-dimensional data, a certain point on the back portion of the subject can be identified on the x-y coordinate, and the depth (that is, the height) on the point with respect to the three-dimensional sensor 3 can be identified on the z-coordinate. In the present embodiment, the three-dimensional data obtaining unit 11 obtains the three-dimensional data of the back portion of the subject based on information measured by the three-dimensional sensor 3. However, the three-dimensional data obtaining unit 11 can also read out the preliminarily recorded three-dimensional data of the back portion from the local storage unit 10. In addition, in a case where the central processing device 2 is connected to another server device via a network, such as the Internet or an intranet, the three-dimensional data obtaining unit 11 may obtain the preliminarily recorded three-dimensional data of the back portion from this server device.

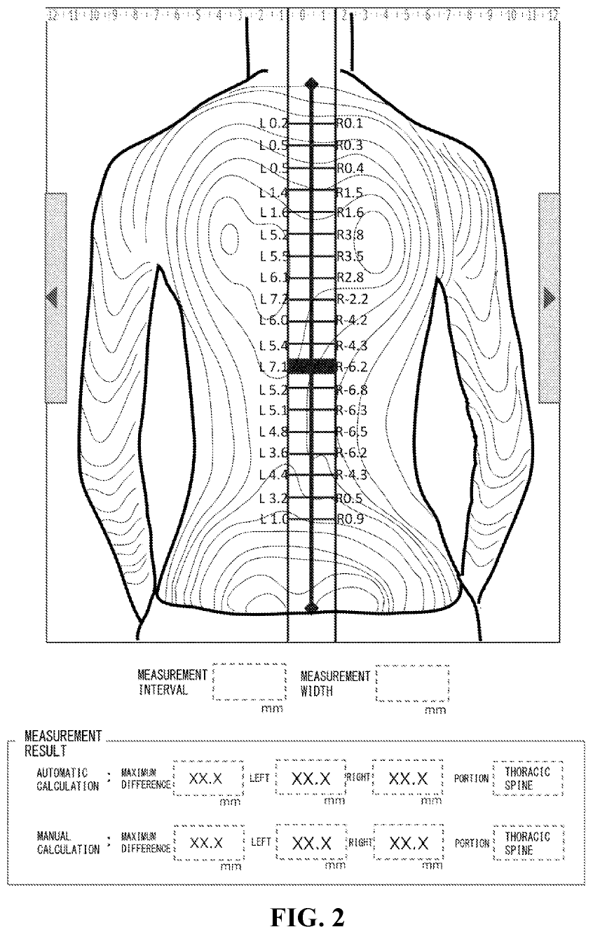

A moire visual image converting unit 12 converts the three-dimensional data obtained by the three-dimensional data obtaining unit 11 into a moire visual image 12. An example of the moire visual image is illustrated in FIG. 2. That is, the moire visual image converting unit 12 refers to the three-dimensional coordinates of the back portion of the subject and, by superimposingly displaying contour lines (moire fringes) connecting points having z-coordinate values that belong in the same levels (predetermined threshold value ranges) in the captured image of the back portion of the subject, generates a moire visual image as indicated in FIG. 2. The generated moire visual image here is stored in the storage unit 10.

An integrated image creating unit 13 creates an integrated image associating the moire visual image created by the moire visual image converting unit 12 with the three-dimensional data obtained by the three-dimensional data obtaining unit 11. That is, while the moire visual image itself does not include three-dimensional coordinate data, the integrated image creating unit 13 associates the three-dimensional data that has become the base of the moire visual image with this moire visual image, and thereby matches the xyz three-dimensional coordinates to the respective points in the moire visual image. Accordingly, for example, by designating a certain point on the moire visual image, an integrated image that allows obtaining an xyz-coordinate of the point can be obtained.

A display unit 14 causes the integrated image created by the integrated image creating unit 13 to be displayed on the display screen of the display 4. Accordingly, as illustrated in FIG. 2, the integrated image associating the moire visual image with the three-dimensional data is displayed as a back portion image. The operator confirms this integrated image (moire visual image) by visual observation while performing designation operations of plots, a measurement interval, and a measurement width, which will be described next.

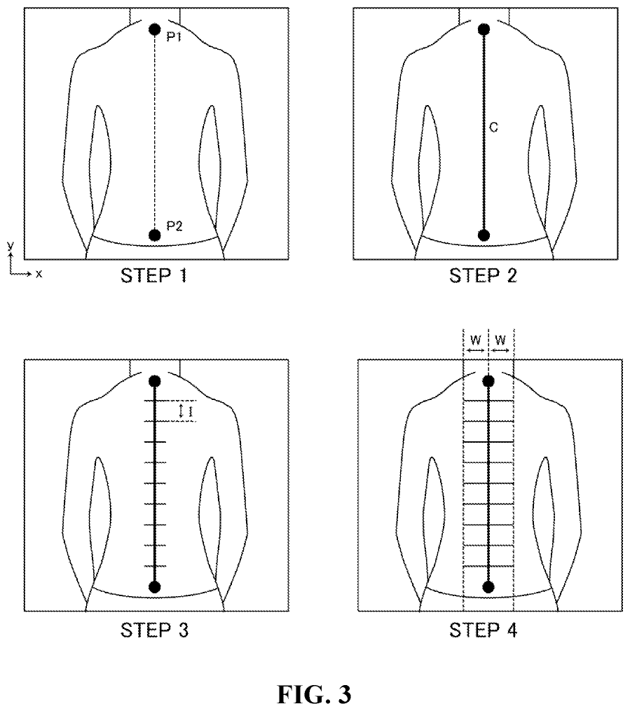

Subsequently, the designation operations of the plots, the measurement interval, and the measurement width will be described. FIG. 3 schematically illustrates back portion images (integrated images) displayed on the display 4. As described above, the moire visual images are superimposingly displayed in the back portion images, but here the moire fringes are omitted to avoid complicating the drawing.

As illustrated in Step 1 of FIG. 3, the operator designates a plurality of plots P1, P2 in the back portion image via the input device 5. A plot designating unit 15 of the central processing device 2 accepts the designation of the plots P1, P2 from the input device 5. The plots need to be on at least two positions, but can be designated on three positions or more, or four positions or more. The plots on two positions are basically designated near the upper end (P1) and near the lower end (P2) of the spine of the subject. The plots P1, P2 on two positions can be arbitrarily designated by the operator. However, for example, a function of assisting the plot P1 and the plot P2 to be aligned on the x-axis of the back portion image that is an x-y plane may be provided in the plot designating unit 15. In addition, the plot designating unit 15 may further accept designation of one or a plurality of intermediary plots between these plots P1, P2 on the two positions near the upper end and near the lower end. Alternatively, the plot designating unit 15 may have a function of scanning the obtained three-dimensional data, extracting points having z-coordinates indicating the depth that are equal to or less than a predetermined value as the outline of the back portion of the subject, among these, obtaining the respective coordinates on the left and right ends of the outline on the upper side in the y-axis direction (such as the neck or shoulder line) and on the left and right ends of the outline on the lower side (such as the waist line), and automatically designating the plots P1, P2 on two positions as midpoints of these left and right ends.

Next, as illustrated in Step 2 of FIG. 3, a center line creating unit 16 creates a rectilinear center line C in the back portion image so as to pass through the plots P1, P2 whose designation has been accepted by the plot designating unit 15. This center line C is assumed to be a line along the spine of the subject in the back portion image. However, depending on the case of scoliosis, the center line C and the actual spine may possibly be displaced. Note that, as long as the center line C passes through the plots P1, P2, it may be a line inclined with respect to the x-axis of the back portion image as an x-y plane, or may be a perpendicular line in parallel to the x-axis of the back portion image. In addition, as described above, in a case where the plot designating unit 15 has a function of assisting the plot P1 and the plot P2 to be aligned on the x-axis of the back portion image, the center line C is always a perpendicular line in parallel to the x-axis of the back portion image.

Next, as illustrated in Step 3 of FIG. 3, the operator designates an arbitrary measurement interval I along the center line C via the input device 5. A measurement interval designating unit 17 of the central processing device 2 accepts the designation of the measurement interval I from the input device 5. In the present embodiment, the measurement interval I can be designated by, for example, inputting an arbitrary length (mm or cm). In addition, by designating one measurement interval I on the center line C, each of the measurement intervals I is set to be an interval the same as this. However, the respective measurement intervals I may be able to be set one by one individually, or may be divided into a plurality of groups to be set for each group. In addition, as a designation method of the measurement interval I, for example, input of the number of intervals (N) disposed on the center line C may be obtained. In this case, the measurement interval I can be automatically calculated by dividing the length of the center line C by the input number of intervals (N).

Next, as illustrated in Step 4 of FIG. 3, the operator designates an arbitrary measurement width W toward the intersecting directions (specifically, perpendicular directions) of the center line via the input device 5. A measurement width designating unit 18 of the central processing device 2 accepts the designation of the measurement width W from the input device 5. In the present embodiment, the measurement width W has equally spaced intervals on the right and left, and all the measurement intervals have the same measurement width W. Therefore, the operator only needs to input one measurement width W. However, the measurement width W may be able to be set one by one individually for each measurement interval, or the respective intervals may be divided into a plurality of groups, and the measurement width may be able to be set for each group.

Steps 1 to 4 illustrated in FIG. 3 are processes accompanying the input operation of the operator. In the processes after this, the coordinate value of each measurement point and the like is automatically calculated based on the plots P1, P2, the measurement interval I, and the measurement width W input in the respective steps hitherto.

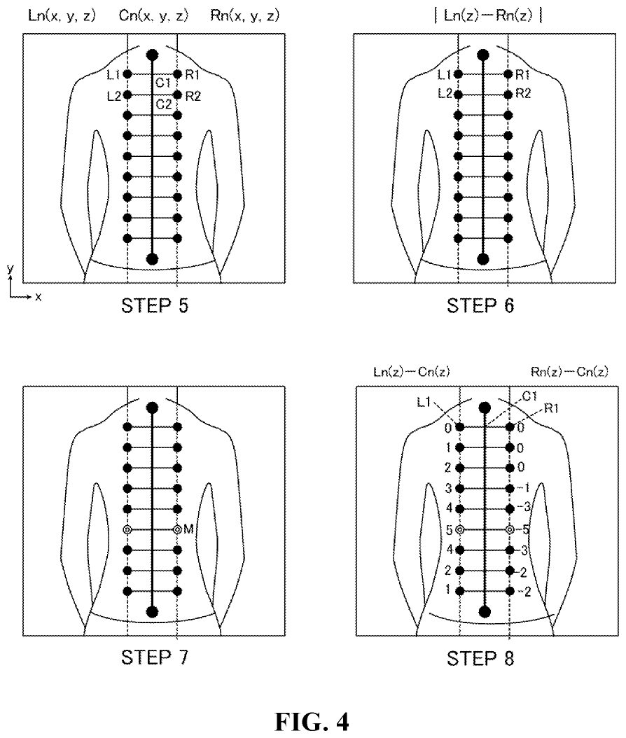

As illustrated in Step 5 of FIG. 4, a measurement point coordinate obtaining unit 19 of the central processing device 2 obtains three-dimensional coordinates (xyz-coordinates) of respective measurement points L, R that are apart from the center line on both the right and left sides by the measurement width W at every measurement interval I. Especially, in the present invention, a depth coordinate (z-coordinate) of each measurement point is necessary. Note that, in FIG. 4, the measurement point on the left side of the center line C is indicated by reference sign L, the measurement point on the right side is indicated by reference sign R, and numbers such as L1 and L2 or R1 and R2 are assigned in the order from the upper measurement points. In addition, for convenience, the n-th right and left measurement points from the top are respectively assumed to be Ln and Rn. As described above, the integrated image displayed as the back portion image includes three-dimensional data. Therefore, by identifying the measurement points L, R in the back portion image, the three-dimensional coordinate of each measurement point L, R can be obtained from the three-dimensional data included in this integrated image. In addition, the measurement point coordinate obtaining unit 19 may obtain an xyz-coordinate of an intermediate point on the center line C between the paired right and left measurement points L, R. In FIG. 4, the intermediate points of the center line C are assigned reference numerals, such as C1 and C2, in order from the top. The coordinate value of each measurement point obtained by the measurement point coordinate obtaining unit 19 is recorded in the storage unit 10.

Next, as illustrated in Step 6 of FIG. 4, a right-left difference calculating unit 20 calculates a difference (referred to as “right-left difference”) between the depth coordinates (z-coordinates) on the paired right and left measurement points L, R disposed at symmetrical positions of the center line C at every measurement interval I. The right-left difference calculating unit 20 only needs to, for example, obtain an absolute value of the difference between the right and left measurement points L, R by the formula: |Ln(z)−Rn(z)|.

Next, as illustrated in Step 7 of FIG. 4, a curved portion identifying unit 21 compares the right-left difference between the right and left measurement points L, R calculated by the right-left difference calculating unit 20 at each measurement interval, and thereby identifies a position where this right-left difference is at its maximum as a curved portion M where a curve degree of the back portion of the subject is high. Thus, identifying the curved portion M where the right-left difference is at its maximum is one process that is important in the examination of scoliosis. By automatizing this process, the interpretation of the back portion image by the operator can be efficiently assisted.

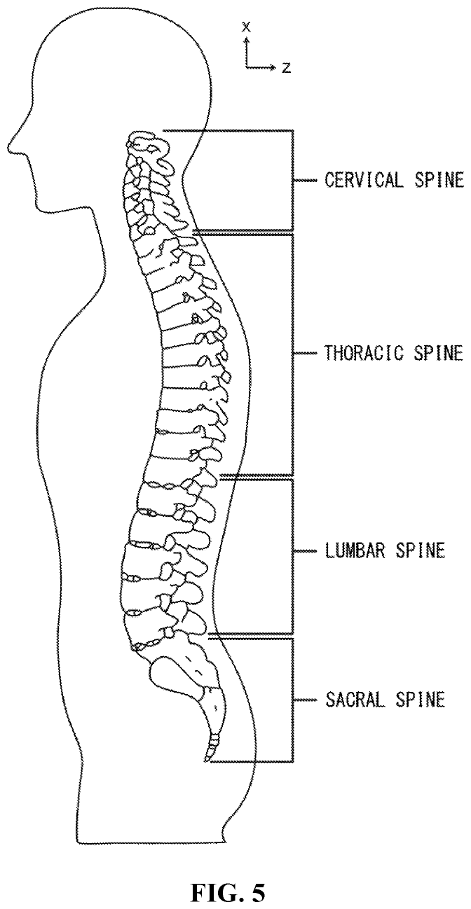

In addition, as illustrated in FIG. 5, the spine of a human body is generally sectioned into regions of the cervical spine, the thoracic spine, the lumbar spine, and the sacral spine. Therefore, also in the present embodiment, the center line C in the back portion image is sectioned into four regions (cervical spine, thoracic spine, lumbar spine, and sacral spine) according to the regions of the spine of a human body. In this case, the curved portion identifying unit 21 may identify the curved portion M as described above, and then determine in which region of the center line this curved portion M belongs. In the examination of scoliosis, it is diagnosed in which region the curved portion M belongs, and by automatizing this process, the interpretation of the back portion image by the operator can be assisted further efficiently.

Next, as illustrated in Step 8 of FIG. 4, a height difference calculating unit 22 calculates the height differences between the depth coordinates of the respective paired right and left measurement points disposed in symmetrical positions across the center line C, and the depth coordinate of the intermediate point on the center line between the right and left measurement points at every measurement interval. For example, it is assumed that the depth coordinate of the left side measurement point Ln is Ln(z), the depth coordinate of the right side measurement point Rn is Rn(z), and the depth coordinate of the intermediate point Cn positioned between these right and left measurement points Ln, Rn is Cn(z). In this case, the height difference calculating unit 22 performs a computation of the formula: Ln(z)+Cn(z) and the formula: Rn(z)−Cn(z). Accordingly, information on what degree of height difference the respective right and left measurement points have compared with the intermediate point is quantified.

As illustrated in the block diagram of FIG. 1, the information obtained by the right-left difference calculating unit 20, the curved portion identifying unit 21, and the height difference calculating unit 22 is transmitted to the display unit 14, and output from this display unit 14 to the display 4. In FIG. 2, an exemplary screen displayed on the display 4 is illustrated. First, among the right-left differences between the right and left measurement points obtained by the right-left difference calculating unit 20, the curved portion where the right-left difference is at its maximum is highlighted by a bold line or the like in the back portion image. Then, the value of the right-left difference is displayed in a column “Automatic Calculation: Maximum Difference: XX.X mm”. In addition, the region in which the curved portion identified by the curved portion identifying unit 21 belongs is displayed in a column “Portion: thoracic spine” (“thoracic spine” is an exemplification). Further, the height differences between the right and left measurement points and the intermediate point obtained by the height difference calculating unit 22 are displayed in association with the respective measurement points in the back portion image. In addition, among the height differences obtained by the height difference calculating unit 22, the value of the height difference between the left side measurement point and the right side measurement point equivalent to the curved portion is displayed in columns “Left: XX.X mm” and “Right: XX.X mm”. Thus, on the display 4, along with the back portion image of the subject to which the moire fringes are given, various information useful for examination of scoliosis is displayed like a list. In addition, in a case where a user has determined that the curved portion where the automatically calculated right-left difference is at its maximum is displaced, it is possible to shift the bold line up and down in the y-axis direction to finely adjust it by the operation of the input device 5. The result is displayed in a column “Manual Calculation: Maximum Difference: XX.X mm”.

FIG. 5 schematically illustrates a difference between the prior art (Patent Document 1: WO2013/081030) and the present invention. As indicated in FIG. 5(a), in the prior art, projected peaks positioned on both the right and left sides of the spine in the back portion of the subject are identified, and the height difference h between these peak positions is calculated. However, as indicated in FIG. 5(b), in a case where a projected peak does not exist on both the right and left sides of the spine (center line C), the prior art does not function. In contrast to this, in the present invention, as indicated in FIG. 5(b), the center line C can be arbitrarily designated, and the measurement width W in both the right and left side directions from this center line C can also be arbitrarily designated. Subsequently, in the present invention, the height difference h (that is, the right-left difference between the depth coordinates) between the right and left measurement points Ln, Rn identified by the measurement width W is obtained. Therefore, the present invention does not depend on projected peaks, and even in a case where a peak does not exist on both the right and left sides of the center line C, the height difference h between the right and left measurement points Ln, Rn can be obtained. Therefore, the interpretation assistance apparatus 1 of the present invention is generally applicable to any case of scoliosis.

In addition, the interpretation assistance apparatus 1 of the present invention has a function of collectively processing a plurality of back portion images as interpretation targets in a case where these plurality of back portion images exist. Note that, while the plurality of back portion images as the interpretation targets are all preferred to be those of the same subject, there is no problem in the process even if a back portion image of a different subject is included. First, the display unit 14 displays a representative back portion image (representative image) among the plurality of back portion images on the display 4. The plot designating unit 15 preliminarily accepts designation of a plurality of plots common to the plurality of back portion images as the interpretation targets in the representative image. The measurement interval designating unit 17 preliminarily accepts designation of a measurement interval common to the plurality of back portion images. The measurement width designating unit 18 preliminarily accepts designation of a measurement width common to the plurality of back portion images. The center line creating unit 16 creates a center line that passes through the designated plurality of plots in each of the plurality of back portion images. Then, the measurement point coordinate obtaining unit 19 collectively obtains the three-dimensional coordinate values (especially the depth coordinates) of the measurement points in each of the plurality of back portion images. Subsequently, for the right-left difference calculating unit 20, the curved portion identifying unit 21, and the height difference calculating unit 22, processes similar to those described above are performed on each of the plurality of back portion images as the interpretation targets. Thus, by accepting designation of the plots, the measurement interval, and the measurement width common to the plurality of back portion images, and collectively obtaining the coordinate values of the measurement points in the respective back portion images, the plurality of back portion images can be simultaneously processed by few input operations. Accordingly, interpretation processing time can be substantially shortened.

In the present application, the embodiments of the present invention have been described above by referring to the drawings to express the contents of the present invention. However, the present invention is not limited to the embodiments described above, and includes changed embodiments and improved embodiments obvious to those skilled in the art based on the matters described in the present application.

### DESCRIPTION OF REFERENCE SIGNS

1. . . . interpretation assistance apparatus
2. . . . central processing device
3. . . . three-dimensional sensor
4. . . . display
5. . . . input device
10. . . . storage unit
11. . . . three-dimensional data obtaining unit
12. . . . moire visual image converting unit
13. . . . integrated image creating unit
14. . . . display unit
15. . . . plot designating unit
16. . . . center line creating unit
17. . . . measurement interval designating unit
18. . . . measurement width designating unit
19. . . . measurement point coordinate obtaining unit
20. . . . right-left difference calculating unit
21. . . . curved portion identifying unit
22. . . . height difference calculating unit

What is claimed is:

1. An interpretation assistance apparatus of a back portion image, comprising:
a center line creating unit that creates a center line in a back portion image of a subject;
a measurement interval designating unit that accepts designation of an arbitrary measurement interval along the center line;
a measurement width designating unit that accepts designation of an arbitrary measurement width toward intersecting directions of the center line; and
a measurement point coordinate obtaining unit that obtains depth coordinates of respective measurement points that are apart from the center line on both right and left sides by the measurement width at every measurement interval.

2. The interpretation assistance apparatus according to claim 1, further comprising
a plot designating unit that accepts designation of an arbitrary plurality of plots in the back portion image, wherein
the center line creating unit creates the center line so as to pass through the plots in the back portion image.

3. The interpretation assistance apparatus according to claim 1, further comprising
a right left difference calculating unit that calculates a right-left difference between depth coordinates of paired right and left measurement points arranged across the center line at every measurement interval.

4. The interpretation assistance apparatus according to claim 3, further comprising
a curved portion identifying unit that identifies a position where the right-left difference is at its maximum as a curved portion where a curve degree of a back portion of the subject is high by comparing the right-left difference at each measurement interval.

5. The interpretation assistance apparatus according to claim 1, further comprising
a height difference calculating unit that calculates height differences between depth coordinates of paired right and left measurement points arranged across the center line at every measurement interval and a depth coordinate on the center line between the right and left measurement points.

6. The interpretation assistance apparatus according to claim 1, further comprising:
a three-dimensional data obtaining unit that obtains three-dimensional data of a back portion of the subject;
a moire image converting unit that converts the three-dimensional data into a two-dimensional moire image;
an integrated image creating unit that creates an integrated image associating the three-dimensional data with the moire image; and
a display unit that displays the integrated image as the back portion image on a display screen.

7. The interpretation assistance apparatus according to claim 4, wherein
the center line is sectioned into regions corresponding to a cervical spine, a thoracic spine, a lumbar spine, and a sacral spine of the subject, and
the curved portion identifying unit identifies the curved portion, and subsequently further determines to which region of the center line the curved portion belongs.

8. The interpretation assistance apparatus according to claim 2, wherein
in a case where a plurality of the back portion images of the subject exist,
the plot designating unit preliminarily accepts designation of the plots common to the plurality of images,
the measurement interval designating unit preliminarily accepts designation of the measurement interval common to the plurality of images,
the measurement width designating unit preliminarily accepts designation of the measurement width common to the plurality of images,
the center line creating unit creates the center line that passes through the plots in each of the plurality of images, and
the measurement point coordinate obtaining unit collectively obtains the depth coordinates in each of the plurality of images.

9. A program for causing a computer to function as the interpretation assistance apparatus according to claim 1.

10. An interpretation assistance method of a back portion image, comprising:
a step of creating a center line that passes through the plots in a back portion image of a subject;
a step of accepting designation of an arbitrary measurement interval along the center line;
a step of accepting designation of an arbitrary measurement width toward intersecting directions of the center line; and
a step of obtaining depth coordinates of respective measurement points that are apart from the center line on both right and left sides by the measurement width at every measurement interval.
